(19) United States
(12) Patent Application Publication
(10) Pub. No.: US 2006/0263402 A1
(43) Pub. Date: Nov. 23, 2006

(54) Oil encapsulation

(75) Inventors: George Endel Deckner, Cincinnati, OH (US); Michael Jude LeBlanc, Cincinnati, OH (US); Jiten Odhavji Dihora, Hamilton, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/437,162

(22) Filed: May 19, 2006

Related U.S. Application Data
(60) Provisional application No. 60/682,600, filed on May 19, 2005.

(51) Int. Cl. A61K 8/02

(52) U.S. Cl. 424/401

(57) ABSTRACT

According to the invention, a solid encapsulate is provided comprising: (a) an oil phase; (b) a water-soluble emulsification polymer, wherein a 0.1 %wt aqueous solution of the water-soluble emulsification polymer has a surface tension of 15-60 mN/m (15-60 dynes/cm) when measured at 25° C.; (c) a water-soluble film-forming polymer; wherein the water-soluble emulsification polymer is different from the water-soluble film-forming polymer.

### CROSS REFERENCE TO RELATED APPLICATION

This Application claims the benefit of U.S. Provisional Application No. 60/682600, filed May 19, 2005.

### FIELD OF THE INVENTION

The present application relates to encapsulates comprising an oil phase a water-soluble emulsification polymer and a water-soluble film-forming polymer, to a method for making the encapsulates and to products comprising the encapsulates.

### BACKGROUND OF THE INVENTION

It is known to encapsulate hydrophobic active ingredients, such as perfumes, in other materials, such as gums, cyclic oligosaccharides and starches, in order, for example to delay release of the encapsulated materials—reference is made, for example, to EP 0 303 461. Thus encapsulated active ingredients may be incorporated into any number of products to achieve the benefit of delayed release—examples of such products include cosmetic products, such as fragrances, powders and deodorants; fabric treatment products, such as washing powders and fabric softening sheets and wipe products, which may have cosmetic or hygiene applications (for example in baby-care products).

For a number of reasons, starches are often used to encapsulate active ingredients: in the first place, starches are safe, mild and environmentally friendly naturally derived ingredients, being found in corn, wheat, rice and potatoes, for example. Their use thus meets an increasing consumer preference for products comprising safe, naturally derived materials. Secondly, starches may bestow advantageous sensory properties, such as improved lather, enriched texture, superior feel on application and improved after application feel, to consumer products, especially in the cosmetic area.

On the other hand, raw, unmodified naturally derived starch may have poor aesthetics and functionality. It is therefore normal to modify it: such modification may be physical—it is common to “pre-gelatinise” starch to render it dispersible in cold water and cold-processable. It is also standard to chemically modify starches used for encapsulation purposes to render them more hydrophobic, increase their viscosity stability and their tolerance of high stress and shear. The hydrophobic modification can be time consuming, complicated and costly. It would therefore be advantageous to find a straightforward way of encapsulating active ingredients in starch that has not been hydrophobically modified.

### SUMMARY OF THE INVENTION

According to a first aspect of the invention, a solid encapsulate is provided comprising:

(a) an oil phase;

(b) a water-soluble emulsification polymer, wherein a 0.1 %wt aqueous solution of the water-soluble emulsification polymer has a surface tension of 15-60 mN/m (15-60 dynes/cm) when measured at 25° C.

(c) a water-soluble film-forming polymer;

wherein the water-soluble emulsification polymer is different from the water-soluble film-forming polymer.

According to a second aspect of the invention, a method is provided for the manufacture of the solid encapsulate according to the first aspect of the invention, comprising the steps of:

(A) forming a high internal phase (HIP) oil-in-water emulsion comprising, by weight of the HIP phase emulsion:
(i) from 0.25% to 7% water-soluble emulsification polymer;
(ii) more than 60%, preferably from 70% to 90% oil phase; and
(iii) water;

(B) forming an aqueous solution of the water-soluble film-forming polymer comprising from 5% to 40% water-soluble film-forming polymer by weight of the aqueous solution;

(C) mixing the HIP emulsion of step A with the aqueous solution of step B to form an aqueous pre-mixture;

(D) drying the aqueous pre-mixture of step C to form solid encapsulate comprising less than or equal to 10% water by weight of the encapsulate.

Solid encapsulate obtainable according to the method of the second aspect of the invention also forms part of the present invention.

According to a third aspect of the invention, a laundry product, especially a granulated detergent or a fabric softening sheet, is provided comprising from 0.01% to 30%, preferably from 0.10% to 12%, more preferably 0.10% to 5% by weight of the encapsulate of the first aspect of the invention.

According to a fourth aspect of the invention, a personal care product, especially a bar soap or an antiperspirant composition, is provided comprising from 0.01% to 30%, preferably from 0.10% to 12%, more preferably 0.10% to 5% by weight of the encapsulate of the first aspect of the invention.

While the specification concludes with claims which particularly point out and distinctly claim the invention, it is believed the present invention will be better understood from the following description of preferred embodiments taken in conjunction with the accompanying drawing.

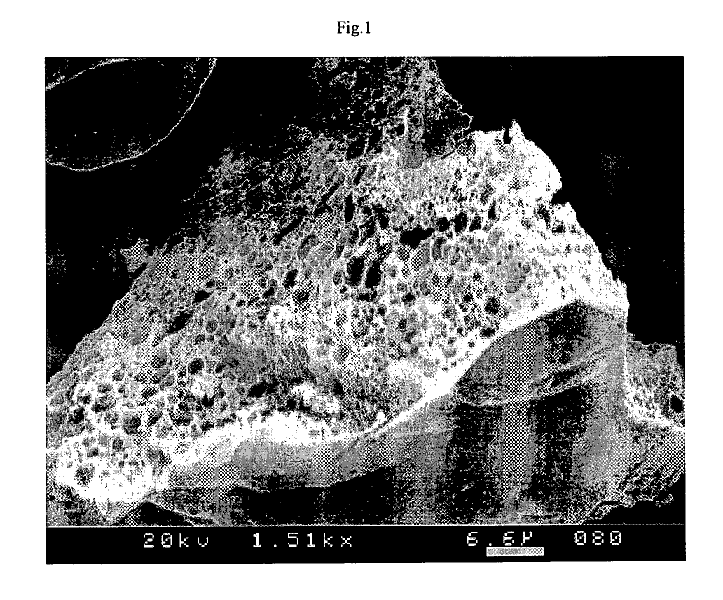

FIG. 1 is scanning electron microscope (SEM) image of a particulate encapsulate according to the invention, that has been broken open.

### DETAILED DESCRIPTION OF THE INVENTION

All weights, measurements and concentrations herein are measured at 25° C. on the composition in its entirety, unless otherwise specified.

Unless otherwise indicated, all percentages of compositions referred to herein are weight percentages of the total composition (i.e. the sum of all components present) and all ratios are weight ratios.

Unless otherwise indicated, all polymer molecular weights are number average molecular weights.

Unless otherwise indicated, the content of all literature sources referred to within this text are incorporated herein in full by reference.

Except where specific examples of actual measured values are presented, numerical values referred to herein should be considered to be qualified by the word “about”.

The present inventor has surprisingly discovered that an oil phase may be encapsulated within a water-soluble film-forming polymer, such as an unmodified starch, by formulating the oil phase as a high internal phase oil-in-water emulsion (O/W HIP or HIPE) using a defined water-soluble emulsification polymer to stabilise the emulsion, then mixing the HIP emulsion with a water-soluble film-forming polymer, such as a hydrolyzed starch. Following combination, the mixture is dried, for example by spray-drying or extrusion, to form a solid encapsulate comprising oil phase, water-soluble emulsification polymer and water-soluble film-forming polymer. As discussed below, it is desirable that the solid encapsulate be substantially anhydrous.

Encapsulates according to the first aspect of the invention comprise an oil phase. The oil phase may comprise any water immiscible material that is liquid at ambient conditions; any material that is solid at ambient conditions, has a melting temperature of less than 100° C. and melts to form a water immiscible liquid; mixtures of such materials.

As used herein in relation to the oil phase, the term “water immiscible” includes materials having a Hildebrand Solubility Parameter of around 5-12 calories/cc (209-502 kJ/m²). The solubility parameter is defined as the sum of all attractive forces radiating out of a molecule. The total Van der Waals force is called the Hildebrand Solubility Parameter and can be calculated using Hildebrand's equation using boiling point and MW data. Methods and a computer program for calculating the Hildebrand Solubility Parameter are disclosed by C. D. Vaughan in J. Cosmet. Chem. 36, 319-333 (September/October 1985). Preferably, the term “water immiscible” relates to materials which additionally have a solubility of less than 0.1 % in deionised water at STP.

Materials comprised within the oil phase may have any polarity and may be selected from the group consisting of aliphatic or aromatic hydrocarbons, esters, alcohols, ethers, carbonates, fluorocarbons, silicones, fluorosilicones, oil-soluble active agents, such as vitamin E and its derivatives, and mixtures thereof.

Solid materials that may be present in the oil phase include waxes. As used herein, the term “wax” includes natural and synthetic waxes. The class of natural waxes includes animal waxes, such as beeswax, lanolin, shellac wax and Chinese insect wax; vegetable waxes, such as carnauba, candelilla, bayberry and sugar cane; mineral waxes, such as ceresin and ozokerite; petrochemical waxes, such as microcrystalline wax and petrolatum. The class of synthetic waxes includes ethylenic polymers and polyol ether-esters, chlorinated naphthalenes and Fischer-Tropsch waxes. For more details, please refer to see Römpp Chemie Lexikon, Georg Thieme Verlag, Stuttgart, 9^{th} Edition, 1995 under “Wachse”.

Advantageously, materials comprised within the oil phase, including the melted waxes, have a viscosity in the range from 0.005 to 15,000 cm²/s (0.5 to 1,500,000 cst), preferably from 0.005 to 10,000 cm²/s (0.5 to 1,000,000 cst), more preferably from 0.005 to 3500 cm²/s (0.5 to 350,000 cst). This viscosity is measured at 25° C. by means of a Brookfield RVT Heliopath Viscometer fitted with a TE Spindle rotating at 5 rpm (if the material is not liquid at 25° C. then the measurement is taken at the temperature at which it becomes fully liquefied).

The oil phase according to the present invention has a dielectric constant in the range 2 to 14, when measured at 20° C. Preferably, dielectric constant of the oil phase is from 3 to 10, more preferably from 6 to 10. The higher the dielectric constant, the more polar the material tends to be.

Examples of oils having a dielectric constant in this range are provided in Table 1.

TABLE 1

| Perfume Oil | Dielectric Constant |
|---|---|
| Citral | 13.80 |
| Beta Gamma Hexenol | 13.70 |
| Benzyl Alcohol | 13.00 |
| Phenyl Ethyl Alcohol | 12.16 |
| Ionone Gamma Methyl | 10.03 |
| Ethyl 2-Methyl Butyrate | 9.48 |
| Ethyl Methyl Phenyl Glycidate | 9.48 |
| Helional | 8.49 |
| Melonal | 8.22 |
| Citronellol | 7.61 |
| Floralozone | 7.10 |
| Syringa Aldehyde | 7.05 |
| Cis Hexenyl Salicylate | 6.94 |
| Decyl Aldehyde | 6.93 |

According to this embodiment, the oil phase may comprise one or more oils, provided that the dielectric constant of the oil phase is in the defined range. The oil phase may comprise from 20 to 60%, preferably from 30 to 50% by weight of the encapsulate.

Encapsulates according to the first aspect of the invention comprise a water-soluble emulsification polymer. A 0.1 %wt aqueous solution of water-soluble emulsification polymer has a surface tension of 15-60 mN/m (15-60 dynes/cm) when measured at 25° C. Within this surface tension range, beneficial emulsification properties are observed.

As used herein, the term “water-soluble” when used in relation to the emulsification polymer means an emulsification polymer having a water solubility as defined in the “Solubility Test Method” hereinbelow.

As used herein, the term “emulsification polymer” includes polymers that have surface-active properties and is not dependent upon a particular chemistry—polymers having widely differing chemistries may be employed.

The water-soluble emulsification polymers according to the invention advantageously have a molecular weight of at least 1000 Daltons, since below this level, the resulting encapsulates may have poor functionality, such as skin feel and poor stability. Skin feel and stability improve with increasing molecular weight and it is preferred that the water-soluble emulsification polymers according to the invention have a molecular weight above 7500 Daltons, more preferably above 9000 Daltons and, more preferably still, above 10,000 Daltons.

The molecular weight of the emulsification polymers advantageously does not exceed 100 kiloDaltons; above that point, especially at the concentrations of emulsification polymer that one would typically use during processing when the internal oil phase is present at levels above 80% by weight of the emulsion, the viscosity of the aqueous phase may reach a level that hinders emulsification.

Non-limiting examples of water-soluble emulsification polymers which may be employed according to the invention include: alkylated polyvinylpyrrolidone, such as butylated polyvinylpyrrolidone commercialised as “Ganex P904” by ISP Corp.; terephthalate polyesters, including polypropylene glycol terephthalate, such as the product commercialised as “Aristoflex PEA” by Clariant A.G.; mono alkyl esters of poly(methyl vinyl ether/maleic acid) sodium salt, including mono butyl ester of poly(methyl vinyl maleic acid sodium salt) such as included in the product commercialised as “EZ Sperse” by ISP Corp; isobutylene/ethylmaleimide/hydroxyethyl copolymer, such as included in the product commercialised as “Aquafix FX64” by ISP Corp.; (3-dimethylaminopropyl)-methacrylamide/3-methacryloylamidopropyl-lauryl-dimthyl-ammonium chloride, such as included in the product commercialised as Styleze W20 by ISP Corp.; peg-12 dimethicone, such as the product commercialised as “DC 193” by Dow Corning Corp.

Highly advantageously, the water-soluble film-forming polymer does not comprise any ethylene oxide group. More advantageously, the water-soluble film-forming polymer is non-alkoxylated and does not comprise any polyglycerol. This is because, during processing, it may prove difficult to dry the aqueous solution to generate the present encapsulates. The disadvantages of having such moieties present in the water-soluble film-forming polymer are particularly noticeable during spray-drying, in which, in place of a particulate encapsulate a sticky deposit may be formed on the sides of the spray-drier. Without wishing to be bound by theory, it is believed that such ethylene oxide groups in particular, but alkoxylated groups and polyglycerol groups in general may hydrogen bond with water, thereby slowing the rate of water evaporation. Of the above-listed materials, Aristoflex PEA comprises propylene oxide groups, but no ethylene oxide groups and DC193 comprises both ethylene oxide and propylene oxide groups.

As used herein, the term “non-alkoxylated” in relation to the water-soluble emulsification polymers means polymers comprising no alkoxy groups, that is no —OR groups (where R includes alkyl moieties) in the molecule, neither in the polymer backbone, nor as pendants thereto nor elsewhere. As used herein, the term “ethylene oxide” or EO means —OC₂H₄— and “propylene oxide” or PO means —OC₃H₆—.

The water-soluble emulsification polymer may comprise from 0.1 to 12%, preferably from 0.5 to 8%, more preferably from 0.5 to 5% by weight of the encapsulate.

Encapsulates according to the first aspect of the invention comprise a water-soluble film forming polymer, which is different from the water-soluble emulsification polymer. In this regard, the word “different” means that the water-soluble film-forming polymer is not identical to the water-soluble emulsification polymer and preferably it means that the water-soluble film-forming polymer does not belong to the same chemical class as the water-soluble emulsification polymer. In one embodiment, the water soluble film-forming polymer is not a water-soluble emulsification polymer and/or the water-soluble film-forming polymer is not a water-soluble emulsification polymer.

As used herein, the term “water-soluble” when used in relation to the film-forming polymer means a film-forming polymer having a water solubility as defined in the “Solubility Test Method” hereinbelow.

As used herein, the term “film-forming” means in relation to the water-soluble film-forming polymer means that the polymer has the ability to transform from a fluid to a solid state as a result of drying (i.e. the removal of solvent, not limited to water) and/or hardening. More details are provided in Deutsche Norm, DIN 55945 under the definition of “Verfestigung, Filmbildung” and associated definitions.

Advantageously, film-forming polymers according to the invention are not cross-linked and more advantageously, they comprise linear or branched-chain polymers that are not cross-linked. Highly advantageously, film-forming polymers according to the invention have a molecular weight from 1 kiloDalton to 500,000 kiloDaltons, preferably from 1 kiloDalton to 100,000 kiloDaltons.

The film-forming polymers according to the invention comprise no hydrophobically modified starch, since it is an object of the present invention to avoid the use of such materials.

Non-limiting examples of water-soluble film-forming polymers which may be employed according to the invention may include: natural gums such as gum Arabic; dextranized or hydrolyzed starches; polyvinyl alcohol; plant-type sugars such as dextrin and maltodextrin; modified starches such as an ungelatinized starch acid ester of a substituted dicarboxylic acid, which may be selected from the group consisting of succinate starch, substituted succinate starch, linoleate starch, and substituted linoleate starch; mixtures thereof.

The water-soluble film-forming polymer may comprise from 5 to 60%, preferably from 30 to 50% by weight of the encapsulate. Additionally and advantageously, the weight ratio of oil phase to solid water-soluble film-forming polymer in the encapsulate is in the range 1:3 to 2:1. If the amount of oil present is such that the weight ratio of oil phase to solid water-soluble film-forming polymer is less than 1:3, then the encapsulate “shell” around the oil phase may typically be too resistant to external forces and other factors to release the oil phase at an acceptable rate. If, on the other hand, weight ratio of oil phase to solid water-soluble film-forming polymer is less than to 2:1, then the encapsulate may be too unstable to adequately contain the oil phase and may permit its premature release. Preferably weight ratio of oil phase to solid water-soluble film-forming polymer is about 1:1.

Advantageously, the encapsulates according to the first aspect of the invention are anhydrous, that is they comprise no water. However, water remnants are likely to be present even immediately after manufacture as a result of processing limitations and it typically occurs that water will re-enter the encapsulates subsequently, for example during storage. The aqueous phase may not only comprise water, but may also comprise additional water-soluble components, such as alcohols; humectants, including polyhydric alcohols (e.g. glycerine and propylene glycol); active agents such as d-panthenol, vitamin B₃ and its derivatives (such as niacinamide) and botanical extracts; thickeners and preservatives. Advantageously, the aqueous phase does not represent more than 10% by weight of the encapsulate and will typically comprise from 0.001% to 10%, preferably from 0.001% to 5%, more preferably from 0.001% to 2%, still more preferably from 0.001% to 1% by weight of the encapsulate.

The encapsulates according to the invention may take any appropriate physical. In particular, they may take the form of particulates, which particulates will advantageously have a median particle size from 5 μm to 200 μm. With reference to FIG. 1, a particulate encapsulate according to the invention is illustrated, which has been broken open to reveal the interstices. Most of the substance of the particulate that can be seen is formed of film-forming polymer (starch in this instance), the open spaces being filled with oil phase. The emulsification polymer is not visible, but is present at the interface between the film-forming polymer and the oil phase.

The present encapsulates are not limited to the particulate form, however, and may also be applied as coatings on a substrate. In such a case, a structure similar to that shown in FIG. 1 will be present, the only significant difference being that the encapsulate is present as a layer rather than a particulate.

According to a second aspect of the invention, products are provided comprising encapsulates according to the first aspect of the invention. Examples of such products include personal care products, such as bar soaps and antiperspirants; laundry products such as granulated detergents and fabric softening sheets; coatings for diapers and feminine hygiene products.

Personal care, health care and laundry products may comprise from 0.01 to 30%wt, preferably from 0.10 to 12%wt, more preferably 0.10 to 5%wt of the encapsulate according to the first aspect of the invention.

The products according to the second aspect of the invention may comprise additional components. The precise nature of these other components will depend on the nature of the final product, so that it is not possible to present an exhaustive list here. Non-limiting examples of other components include thickeners; solvents; natural and synthetic waxes; emollients; humectants, such as polyhydric alcohols, including glycerine and propylene glycol; pigments, including organic and inorganic pigments; preservatives; chelating agents, antimicrobials and perfumes. Surfactants, such as non-ionic, anionic, cationic, zwitterionic and amphoteric surfactants, may also be present. Where the product comprises a substrate, then the encapsulate (optionally in admixture with one or more of the above-mentioned additional components) may be coated upon the substrate, which substrate may, without limitation, comprise woven or non-woven material or paper.

### ENCAPSULATE MANUFACTURING METHOD

I. Formation of the HIP Emulsion

A high internal phase emulsion is prepared according to the following general method:
1. Aqueous phase components and oil phase components are selected in such quantities to give a high internal phase oil-in-water emulsion on mixing together in step 4, below.
2. The water-soluble emulsification polymer is thoroughly mixed with and solubilized in aqueous phase. The water-soluble emulsification polymer is added in a sufficient amount to comprise from 0.25 to 7%, preferably from 0.25 to 5% by weight of the HIP emulsion formed in step 4, below.
3. The oil phase components are thoroughly mixed together. If waxes or other materials are present, which are solid at room temperature, then this mixing step may also involve heating, as discussed above.
4. The oil phase is slowly added to the aqueous phase with continual mixing to give a high internal phase (HIP) emulsion comprising above 60%, preferably above 70%, more preferably from 70 to 90% oil phase.
   II. Addition of the Water-Soluble Film-Forming Polymer

The water-soluble film-forming polymer is now added to the HIP emulsion. Typically, it is added as an aqueous solution, for example at a concentration from 5% to 40% by weight. As discussed above, the water-soluble film-forming polymer is added in an amount which represents 5%-60%, preferably 30%-50% by weight of the composition on a dry basis. As additionally discussed above, the weight ratio of oil phase to solid water-soluble film-forming polymer is in the range 1:3 to 2:1.

III. Dehydration

A variety of dehydration methods can be applied to the HIP aqueous emulsion system to yield dry particles, including but not limited to vacuum drying, drum drying, freeze drying, thin-film drying (emulsion dispersed onto a water insoluble film and air dried), and spray drying. In addition, one can add the emulsion to an agglomerator (cylindrical vessel fitted with paddle mixers, or high shear choppers) containing a water hydrating material—for example, fine silica gel will absorb water from the aqueous emulsion and yield free flowing powder. Suitable equipment for use in the processes disclosed herein may include paddle mixers, ploughshear mixers, ribbon blenders, vertical axis granulators and drum mixers, both in batch and, where available, in continuous process configurations.

A preferred method for the manufacture of oil encapsulated particles is spray drying. Spray drying may result in very rapid dehydration of the aqueous emulsion (typically this may be achieved in less than one minute), providing minimum loss of volatile oil materials during particle formation. Spray drying may also conveniently provide a means to control the particle size of the finished product.

Typically, during spray drying, an aqueous emulsion is fed to a centrifugal atomizer (spinning disk or spinning wheel), where it is atomized into fine droplets. The speed of the disk is used to manipulate the size of the atomized droplets. Dry, hot air (typically at around 200° C., Dew Point −40° C.) is introduced above the atomizer in a co-current mode (i.e. the air flow moves in the same direction as the product to be dried) to facilitate the rapid dehydration of the atomized droplets. The outlet air temperature is typically maintained between 95° C. to 105° C., depending on the moisture content and wall flexibility desired in the finished particles. The dried particles are then carried by the air to a cyclone (gas/solid separator), where they are collected. The remaining air containing very fine particles not removed by the cyclone is passed to a bag filter or a scrubber.

Measurement Methods

Median Particle Size Test Method

This test method may be used to determine the median particle size of a solid encapsulate according to the first aspect of the invention. The solid encapsulate particle size is determined in accordance with ISO 8130-13, “Coating powders—Part 13: Particle size analysis by laser diffraction.” A suitable laser diffraction particle size analyzer with a dry-powder feeder can be obtained from Horiba Instruments Incorporated of Irvine, Calif., U.S.A.; Malvern Instruments Ltd of Worcestershire, UK; and Beckman-Coulter Incorporated of Fullerton, Calif., U.S.A. The results are expressed in accordance with ISO 9276-1:1998, “Representation of results of particle size analysis—Part 1: Graphical Representation”, Figure A.4, “Cumulative distribution Q₃ plotted on graph paper with a logarithmic abscissa.” The median particle size is defined as the abscissa value at the point where the cumulative distribution (Q₃) is equal to 50 percent.

Solubility Test Method

As used herein in relation to the emulsification polymers and the film-forming polymers, the term “water-soluble” includes polymers fulfilling the following condition: a 1 %wt solution of the polymer in de-ionised water at room temperature gives at least 90% transmittance of light having a wavelength in the range from 455 to 800 nm. Testing was carried out by passing the polymer solution through a standard syringe filter into a 1 cm path length cuvette having a pore size of 450 nm and scanning using an HP 8453 Spectrophotometer arranged to scan and record across 390 to 800 nm. Filtration was carried out to remove insoluble components.

Measurement of Surface Tension

The method used for measuring surface tension of fluid is the so-called “Wilhelmy Plate Method”. The Wilhelmy plate method is a universal method especially suited to establishing surface tension over time intervals. In essence, a vertical plate of known perimeter is attached to a balance, and the force due to wetting is measured. More specifically:
A 0.1%wt aqueous solution of water-soluble emulsification polymer is made up in de-ionised water. The polymer solution is then poured into a clean and dry glass vessel, the solution temperature being controlled at 25° C. The clean and annealed Wilhelmy Plate is lowered to the surface of the liquid. Once the plate has reached the surface the force which is needed to remove the plate out of the liquid is measured.

The equipment used and corresponding settings are as follows:
Device: Krüss Tensiometer K12, manufactured by Krüss GmbH, Borsteler Chausee 85-99a, 22453 Hamburg- Germany (see www.kruess.com).

Plate Dimensions: Width: 19.9 mm; Thickness: 0.2 mm; Height: 10 mm
Measurement Settings: immersion depth 2 mm, Surface Detection Sensitivity 0.01 g, Surface Detection Speed 6 mm/min, Values 10, Acquisition linear, Maximum Measurement Time 60 sec

The plate is immersed in the fluid and the corresponding value of surface tension is read on the display of the device. Instructions can be found in the user manual edited by ,,Krüss GmbH Hamburg 1996” Version 2.1.

Testing the Dielectric Constant of the Polar Oils

Measurements were taken at 20° C. using a Model 870 liquid dielectric constant meter manufactured by Scientifica in Princeton N.J. Readings were taken once equilibrium had been reached (in the rule, it took five to achieve a constant value).

### EXAMPLES

The following examples further describe and demonstrate the preferred embodiments within the scope of the present invention. The examples are given solely for the purpose of illustration, and are not to be construed as limitations of the present invention since many variations thereof are possible without departing from its scope.

### Encapsulation Example 1

Spray-Dried Encapsulated Perfume Oil

| | Material | % wt |
|---|---|---|
| A | Deionised Water | 3.0 |
| | Ganex 904¹ | 1.0 |
| B | “Datura” | 14.0 |
| | Fragrance Oil² | |
| C | PC03-1045³ (50% | 40.0 |
| | solution in water) | |
| D | Deionised water | 40.0 |

¹Butylated poly vinyl pyrrolidone commercialised by ISP.
²Datura fragrance (a combination of perfume oils) has a dielectric constant of 6.65.
³Starch solution (hydrolyzed starch dissolved in deionized water, 33 wt % solids) available from National Starch & Chemical Co. of New Jersey, USA.

Procedure to Make the HIP Oil-in-Water Emulsion

The Ganex P904 is dissolved in water at room temperature until clear to generate pre-mix A.

Fragrance oil B was then slowly added to pre-mix A using a 3-blade turbine mixer attached to a Lightning mixer agitation system at 300 RPM until the emulsion thickens.

Advantageously, the emulsion may additionally be milled for 5 minutes using a Tokuhsa Kika-TK Homogeniser, Mark II, to reduce the average emulsion particle size to below 1 μm.

Mixture With the Water-Soluble Film-Forming Polymer

The mixture of A and B was then added to components C and D and mixed until uniform using a Lightening mixer equipped with a pitch turbine blade. The mixture was then milled for 5 minutes using a Tokuhsa Kika-TK Homogeniser, Mark II.

Dehydration

The mixture was then spray dried using a co-current Niro 6 ft (1.8 m) diameter spray dryer operating with a 2 inch (0.05 m) diameter spinning wheel atomizer, at the following operating conditions: inlet air temperature of 200° C., outlet temperature of 95° C. to 98° C., 80 kg/hr air flow rate, disk speed of 30,000 RPM, and a dryer operating pressure of 0.4 mm H₂O. The particles collected from the dryer have a mean particle size of 50 μm and the following composition:

| Material | % wt |
|---|---|
| Ganex P904 | 2.9 |
| “Datura” Fragrance Oil | 40.0 |
| PC03-1045 | 57.1 |

### Encapsulation Example 2

Spray-Dried Encapsulated Vitamin E

| | Material | % wt |
|---|---|---|
| A | Deionised Water | 3 |
| | EZ Sperse¹ | 1 |
| B | Tocopherol acetate² | 16 |
| C | PC03-1045 | 16 |
| D | Deionised water | 64 |

¹EZ Sperse is a 25% solution of mono butyl ester of poly(methyl vinyl maleic acid sodium salt) and is a copolymer of maleic anhydride and methyl vinyl ether reacted with water/butanol to form a half ester, which is neutralised with sodium hydroxide. EZ Sperse is produced by ISP Corp.
²Tocopherol acetate has a dielectric constant of 3.46 and a solubility parameter of 7.98.

Procedure to Make the HIP Oil-in-Water Emulsion

The EZSperse is dissolved in water at room temperature until clear to generate pre-mix A.

Tocopherol acetate B was then slowly added to pre-mix A using a 3-blade turbine mixer attached to a Lightning mixer agitation system at 300 RPM until the emulsion thickens.

Advantageously, the emulsion may additionally be milled for 5 minutes using a Tokuhsa Kika-TK Homogeniser, Mark II, to reduce the average emulsion particle size to below 1 μm.

Mixture With the Water-Soluble Film-Forming Polymer

The mixture of A and B was then added to components C and D and mixed until uniform using a Lightening mixer equipped with a pitch turbine blade. The mixture was then milled for 5 minutes using a Tokuhsa Kika-TK Homogeniser, Mark II.

Dehydration

The mixture was then spray dried using a co-current Niro 6 ft (1.82 m) diameter spray dryer operating with a 2 inch (0.05 m) diameter spinning wheel atomizer, at the following operating conditions: inlet air temperature of 200° C., outlet temperature of 95° C. to 98° C., 80 kg/hr air flow rate, disk speed of 30,000 RPM, and a dryer operating pressure of 0.4 mm H₂O. The particles collected from the dryer have a mean particle size of 50 μm and the following composition:

| Material | % wt |
|---|---|
| EZSperse | 0.8 |
| Tocopherol acetate | 49.6 |
| PC03-1045 | 49.6 |

### Product Example 1

Invisible Solid Antiperspirant

| Material | % wt |
|---|---|
| Cyclomethicone | 41.05 |
| AlZr Trichlorohydrate Glycine | 24.00 |
| Stearyl Alcohol | 14.50 |
| Phenyl Trimethicone | 12.00 |
| Castor Wax | 3.50 |
| Behenyl Alcohol | 0.20 |
| Petrolatum | 4.00 |
| Lacey Light Fragrance | 1.25 |
| Encapsulated Perfume of Encapsulation | 0.50 |
| Example 1 | |

### Product Example 2

Diaper/Feminine Hygiene Product

The top sheet of a baby diaper/feminine hygiene product is coated and dried using an aqueous solution of the encapsulate (63% water, 37% encapsulate) according to Encapsulation Example 1. Alternatively, 40 mg of the encapsulate of Encapsulation Example 1 may be added as a powder to the absorptive core of the diaper/feminine hygiene product. This provides moisture activated release of fragrance after the baby urinates or menses bleeding has occurred.

### Product Example 3

Laundry Detergent Powder

| | Formulation Examples: | | | | | | | |
|---|---|---|---|---|---|---|---|---|
| | A | B | C | D | E | F | G | H |
| Encapsulated Perfume of | 0.8 | 0.8 | 0.8 | 0.8 | 0.8 | 0.8 | 0.8 | 0.8 |
| Encapsulation Example 1 | | | | | | | | |
| Formulation balance: | | | | | | | | |
| Sodium alkylbenzenesulfonate | 19.99 | 6.10 | 8.19 | 8.48 | 0.07 | 3.41 | 17.45 | 17.45 |
| Sodium alkylsulfate | 1.16 | 12.20 | 5.13 | 6.08 | 15.27 | 13.71 | 0.00 | 0.00 |
| Ethoxylated sodium alkylsulfate | 0.29 | 0.00 | 0.00 | 0.00 | 0.00 | 0.00 | 1.55 | 1.55 |
| Sodium Percarbonate | 6.16 | 6.16 | 0.00 | 3.49 | 2.78 | 4.50 | 11.67 | 3.21 |
| Nonanoyloxybenzenesulfonate | 4.75 | 4.75 | 2.10 | 2.41 | 1.92 | 5.16 | 0.00 | 0.00 |
| Tetraacetylethylenediamine | 0.00 | 0.00 | 0.00 | 0.00 | 0.00 | 0.00 | 2.10 | 2.10 |
| Sodium aluminosilicate hydrate | 13.84 | 12.96 | 25.38 | 27.98 | 32.46 | 32.46 | 14.36 | 12.80 |
| Acrylic/Maleic Acids | 6.35 | 3.36 | 0.00 | 0.00 | 0.00 | 0.00 | 2.30 | 2.30 |
| Copolymer | | | | | | | | |
| Sodium Polyacrylate | 0.00 | 0.00 | 1.51 | 1.53 | 1.74 | 1.18 | 0.00 | 0.00 |
| Sodium Carbonate | 19.55 | 22.25 | 22.48 | 21.47 | 24.11 | 23.33 | 20.60 | 20.60 |
| Sodium Tripolyphosphate | 0.00 | 0.00 | 0.00 | 0.00 | 0.00 | 0.00 | 0.00 | 12.40 |
| Sodium Silicate | 2.43 | 2.47 | 0.00 | 0.00 | 0.00 | 0.00 | 0.00 | 0.00 |
| Sodium | 0.00 | 0.00 | 0.72 | 0.80 | 0.72 | 0.54 | 0.54 | 0.54 |
| diethylenetriaminepentaacetate | | | | | | | | |
| Brightener 15 | 0.17 | 0.17 | 0.00 | 0.11 | 0.08 | 0.12 | 0.12 | 0.12 |
| Brightener 49 | 0.09 | 0.09 | 0.00 | 0.00 | 0.00 | 0.00 | 0.00 | 0.00 |
| Sodium Xylene Sulfonate | 1.81 | 0.00 | 0.00 | 0.00 | 0.00 | 0.00 | 0.00 | 0.00 |
| Polydimethylsiloxane | 0.06 | 0.06 | 0.02 | 0.02 | 0.02 | 0.04 | 0.04 | 0.04 |
| Ethyl Methyl Cellulose | 0.00 | 0.00 | 1.11 | 0.00 | 1.11 | 0.00 | 0.00 | 0.00 |
| Imideazole Epichlorohydrin | 0.00 | 0.00 | 0.15 | 0.00 | 0.15 | 0.00 | 0.00 | 0.00 |
| Savinase active enzyme | 0.054 | 0.054 | 0.015 | 0.010 | 0.015 | 0.021 | 0.021 | 0.021 |
| Carezyme active enzyme | 0.000 | 0.000 | 0.003 | 0.000 | 0.000 | 0.000 | 0.000 | 0.000 |
| Perfume | 0.21 | 0.21 | 0.22 | 0.26 | 0.38 | 0.24 | 0.24 | 0.24 |
| Balance sodium sulfate | | | | | | | | |

Total formulation = 100.00

A procedure for manufacturing such laundry detergent powder compositions is provided in U.S. Pat. No. 5,496,487.

### Product Example 4

Bar Soap

| | A | B | C | D | E | F | G |
|---|---|---|---|---|---|---|---|
| Ingredient | % wt | % wt | % wt | % wt | % wt | % wt | % wt |
| Soap | 80.15 | 77.95 | 80.15 | 72.65 | 80.15 | 77.25 | 80.15 |
| Free Fatty Acid | 5.73 | 5.70 | 5.00 | 3.1 | 5.83 | 5.90 | 5.00 |
| Water | 11.56 | 11.50 | 10.69 | 11.9 | 11.56 | 11.50 | 10.69 |
| Sodium Chloride | 1.11 | 1.10 | 1.11 | 1.10 | 1.11 | 1.10 | 1.11 |
| Titanium Dioxide | 0.25 | 0.25 | 0.25 | 0.25 | 0.25 | 0.25 | 0.25 |
| Perfume | 0.80 | 1.00 | 0.80 | 1.00 | 0.80 | 1.00 | 0.80 |
| Encapsulate of | 0.40 | 2.5 | 2.0 | 10.0 | 0.30 | 3.0 | 2.0 |
| Encapsulation | | | | | | | |
| Example 1 | | | | | | | |

Method of Manufacture: mix perfume and encapsulated fragrance into dried soap noodles in an amalgamator. The material is processed, for example by milling through a 3-roll soap mill, to obtain a homogeneous mixture of perfume & soap flakes. Then the material is processed on a plodder and is stamped into a soap bar.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modification that are within the scope of this invention.

What is claimed is:

1. A solid encapsulate comprising:
(a) an oil phase;
(b) a water-soluble emulsification polymer, wherein a 0.1%wt aqueous solution of the water-soluble emulsification polymer has a surface tension of about 15-60 mN/m when measured at 25° C.;
(c) a water-soluble film-forming polymer;
wherein the water-soluble emulsification polymer is different from the water-soluble film-forming polymer.

2. The solid encapsulate of claim 1, wherein the oil phase comprises materials selected from the group consisting of aliphatic or aromatic hydrocarbons, esters, alcohols, ethers, carbonates, fluorocarbons, silicones, fluorosilicones, and oil-soluble active agents and mixtures thereof.

3. The solid encapsulate of claim 1, wherein the oil phase has a dielectric constant in the range about 2 to about 14 when measured at 20° C.

4. The solid encapsulate of claim 3, wherein the oil phase has a dielectric constant in the range about 3 to about 10 when measured at 20 ° C.

5. The solid encapsulate of claim 1, comprising from about 20 to about 60% oil phase by weight of the encapsulate.

6. The solid encapsulate of claim 5, comprising from about 30 to about 50% oil phase by weight of the encapsulate.

7. The solid encapsulate of claim 1, wherein the water-soluble emulsification polymer has a molecular weight of at least about 1000 Daltons.

8. The solid encapsulate of claim 1, wherein the water-soluble emulsification polymer has a molecular weight of at most about 100 kiloDaltons.

9. The solid encapsulate of claim 1, wherein the water-soluble emulsification polymer is selected from the group consisting of alkylated polyvinylpyrrolidone; terephthalate polyesters; mono alkyl esters of poly(methyl vinyl ether/maleic acid) sodium salt; isobutylene/ethylmaleimide/hydroxyethyl copolymer; (3-dimethylaminopropyl)-methacrylamide/3 -methacryloylamidopropyl-lauryl-dimthyl-ammonium chloride; and peg-12 dimethicone and mixtures thereof.

10. The solid encapsulate of claim 1, wherein the water-soluble emulsification polymer is essentially free of any ethylene oxide groups.

11. The solid encapsulate of claim 1, wherein the water-soluble emulsification polymer is non-alkoxylated.

12. The solid encapsulate of claim 1 comprising from about 0.1% to about 12% water-soluble emulsification polymer by weight of the encapsulate.

13. The solid encapsulate of claim 1, wherein the water-soluble film-forming polymer is selected from the group consisting of a linear or branched chain polymer that is not cross-linked.

14. The solid encapsulate of claim 13, wherein the water-soluble film-forming polymer has a molecular weight from about 1 kiloDalton to about 500,000 kiloDaltons.

15. The solid encapsulate of claim 14, wherein the water-soluble film-forming polymer has a molecular weight from about 1 kiloDalton to about 100,000 kiloDaltons.

16. The solid encapsulate of claim 13, wherein the the water-soluble film-forming polymer is selected from the group consisting of natural gums; dextranized or hydrolyzed starches; polyvinyl alcohol; dextrin and maltodextrin; and ungelatinized starch acid esters of substituted dicarboxylic acids and mixtures thereof.

17. The solid encapsulate of claim 13, comprising from about 5% to about 60% water-soluble film-forming polymer by weight of the encapsulate.

18. The solid encapsulate of claim 13, comprising from about 30% to about 50% water-soluble film-forming polymer by weight of the encapsulate.

19. The solid encapsulate of claim 1, wherein the weight ratio of oil phase to water-soluble film-forming polymer in the encapsulate is in the range about 1:3 to about 2:1.

20. The solid encapsulate according to claim 1 in the form of a particle.

21. The solid encapsulate according to claim 21, having a median particle size from about 5 μm to about 200 μm.

22. Method for the manufacture of the solid encapsulate of claim 1, comprising the steps of:
(A) forming a high internal phase (HIP) oil-in-water emulsion comprising, by weight of the HIP phase emulsion:
(i) from about 0.25% to about 7% water-soluble emulsification polymer;
(ii) more than about 60% oil phase; and
(iii) water;
(B) forming an aqueous solution of the water-soluble film-forming polymer comprising from about 5% to about 40% water-soluble film-forming polymer by weight of the aqueous solution;
(C) mixing the HIP emulsion of step A with the aqueous solution of step B to form an aqueous pre-mixture;
(D) drying the aqueous pre-mixture of step C to form solid encapsulate comprising less than or equal to about 10% water by weight of the encapsulate.

23. A laundry product, especially a granulated detergent or a fabric softening sheet, comprising from about 0.01% to about 30% by weight of the encapsulate according to claim 1.

24. A personal care product, especially a bar soap or an antiperspirant composition, comprising from 0.01% to 30% by weight of the encapsulate according to claim 1.
